(19) United States
(12) Patent Application Publication
(10) Pub. No.: US 2019/0200900 A1
(43) Pub. Date: Jul. 4, 2019

(54) Apparatus for Intraoperative Ligament Load Measurements

(71) Applicant: Wisconsin Alumni Research Foundation, Madison, WI (US)

(72) Inventors: Darryl Gerard Thelen, Madison, WI (US); Jack Anthony Martin, Madison, WI (US); Joshua Daniel Roth, Madison, WI (US)

(21) Appl. No.: 15/857,095

(22) Filed: Dec. 28, 2017

(51) Int. Cl. A61B 5/103; A61B 5/00; A61B 5/11

(52) U.S. Cl. CPC A61B 5/1036; A61B 34/10; A61B 5/1121; A61B 5/4585

(57) ABSTRACT

Axial stress or similar properties in a stressed connective tissue may be assessed during surgery by excitation of the ligament to create a shear wave allowing measurement of the shear wave velocity which has been identified as having a component related to axial stress.

### CROSS REFERENCE TO RELATED APPLICATION

### Background of the Invention

The present invention relates to an apparatus for measuring connective tissue loads (e.g., stress, tension, or the like) and in particular an apparatus suitable for intraoperative use.

Total knee arthroplasty (TKA) is the most common treatment for patients with end-stage osteoarthritis (OA). In TKA, the articulating surfaces of the distal end of the femur and proximal end of the tibia are replaced with prostheses providing a sliding polished metal and plastic interface. During this replacement procedure, the superficial medial collateral ligament (sMCL) and a lateral collateral ligament (LCL), which are located on either side of the knee joint, are preserved to maintain knee stability.

The number of patients undergoing TKA is on the rise with recent predictions that 3.5 million TKAs will be performed annually by 2030 in the United States alone. Unfortunately, 18-25% of these patients are not satisfied because of stiffness, instability, residual pain, and/or functional limitations in the treated knee.

A cause for this dissatisfaction and an indication for revision is improper tensioning of the sMCL and LCL. Surgeons commonly manipulate the tension in the sMCL and LCL through “releases” or adjustments to component alignment in order to stabilize the joint without causing stiffness or pain. Under-tensioned ligaments may lead to instability (especially in mid-flexion) and pain while overly-tensioned ligaments may lead to stiffness and pain.

Despite the well-recognized importance of achieving the correct tension in the soft tissue restraints, accurate adjustment of tension in these structures during surgery is difficult.

### Summary of the Invention

The present invention provides an instrument for measuring ligament forces, for example, stress and tension, in the surgical environment by inducing a shear wave in the ligament and measuring the propagation speed of that shear wave with displaced sensors. Shear wave propagation speed provides an indication of ligament stress, and with knowledge about geometry of the ligament, shear wave propagation speed may be used to deduce tension or the like, which are measures that can provide the physician with real-time information for adjusting ligament tension.

In one embodiment, the device includes a stimulator probe having an electromechanical actuator adapted to apply a transverse stimulation to the connective tissue to generate a shear wave that travels longitudinally along the connective tissue. First and second motion sensors detect transverse motion of the tissue along first and second transverse axes at a first and second respective longitudinal displacement from the stimulator probe. A processing circuit connected to the first and second motion sensors provides a respective first and second transverse motion signal indicating transverse movement of the tissue at the first and second transverse axes over time. The processing circuit compares the first transverse motion signal to the second transverse motion signal to determine a travel time of passage of the shear wave between the first and second locations and processes the travel time to output a value functionally related to the connective tissue stress.

It is thus a feature of at least one embodiment of the invention to provide an intraoperative measure of ligament tension or similar tissue useful during a variety of surgical procedures including but not limited to total knee arthroplasty.

The output may provide a load measure of at least one of longitudinal stress or tension in the connective tissue.

It is thus a feature of at least one embodiment of the invention to permit a variety of different load-type measurements (e.g., stress or tension) as may be appropriate for particular surgical situations.

The processing circuit may include an electronic memory holding a data value indicating a desired preload value for the connective tissue and further including a display indicating a deviation between the desired preload value and actual load measure.

It is thus a feature of at least one embodiment of the invention to provide a clear display suitable for use in the surgical suite that can guide a physician to adjusting ligament tensions acting across the joint.

An electronic memory communicating with the processing circuit may hold a database indexable by index values including at least one of: age, gender, weight, height, ethnicity, and activity level, and may provide a desired preload value for the connective tissue. The device may further include a human machine interface for receiving the index values to search and obtain the desired preload value for the connective tissue.

It is thus a feature of at least one embodiment of the invention to provide the physician with guidance values for adjusting ligaments sensitive to variations in those values among members of the population.

The database may provide geometric information about the connective tissue (for the particular demographic) for the conversion of a load measure of stress into a load measure of tension by the processing circuit.

It is thus a feature of at least one embodiment of the invention to provide geometric information about the ligament useful, for example, in converting various load measures, for example, converting stress to tension.

The database may provide a first and second desired preload value for related connective tissue and the display may simultaneously indicate a deviation between the first desired preload value and a first load measure of a first one of the connective tissue and the deviation between the second desired preload value and a second load measure of a second one of the connective tissue.

it is thus a feature of at least one embodiment of the invention to provide for the storage and display of information facilitating balance between two opposed ligaments, for example.

The first connective tissue may be a superficial medial collateral ligament and the second connective tissue may be a lateral collateral ligament.

It is thus a feature of at least one embodiment of the invention to provide an apparatus for improved total knee arthroplasty.

The processing circuit may include an electronic memory holding a data value indicating a measured load value for a connective tissue structure associated with the joint at multiple positions (for example, full flexion, full extension, and halfway between these states) and further optionally may include a display simultaneously indicating a deviation between the desired preload value and the load measure for the at least two different positions.

It is thus a feature of at least one embodiment of the invention to provide the physician with a ready reference as to how tension changes with joint pose for proper adjustment of the ligaments or joint.

The stimulator probe, first motion sensor, second motion sensor, and at least a portion of the processing circuit may be contained in a housing supportable by the hand of an individual for manual positioning during surgical procedures.

It is thus a feature of at least one embodiment of the invention to provide a compact unit that may be used conveniently in the surgical suite.

The stimulator probe may be attached to the housing with a force-limiting coupling creating a steady-state force between stimulator probe and the connective tissue.

It is thus a feature of at least one embodiment of the invention to allow a handheld unit to be used without unduly influencing the tension measurement caused by variation in the force between the measuring unit and the connective tissue resulting from handheld operation.

The force-limiting coupling may be a spring-biased slide mounting.

It is thus a feature of at least one embodiment of the invention to provide a simple method of minimizing force variations.

The first and second motion sensor may be contact-type motion sensors (e.g., accelerometers) also attached to the housing with a force-limiting coupling.

It is thus a feature of at least one embodiment of the invention to permit the use of contact-type sensors while minimizing the effect of varying transverse contact forces on the measurement being made.

Alternatively, the first and second motion sensors may be non-contact-type motion sensors (e.g., ultrasound, laser vibrometer).

It is thus a feature of at least one embodiment of the invention to minimize contact forces between the measuring instrument and the ligament through the use of non-contact-type sensors.

The stimulator probe, first motion sensor, second motion sensor may be contained in a housing supported by a spacer fitting between a femur and tibia.

It is thus a feature of at least one embodiment of the invention to provide a measurement of connective tissue loads registered to the joint itself.

The spacer may support a first and second set of coupled stimulator probe and motion sensors on both of opposed medial and lateral sides of the spacer for simultaneous measurement of medial and lateral collateral ligaments.

It is thus a feature of at least one embodiment of the invention to permit simultaneous measurement of ligament tension on both lateral and medial sides of a joint.

The device may include a position sensor and a. display indicating a location of the handheld housing with respect to the connective tissue.

It is thus a feature of at least one embodiment of the invention to permit image-guided location of the sensor unit.

The processing circuit may indicate a sequence of targets on the display to which the location of the handheld housing may be matched to provide a set of measurements of the connective tissue in different locations.

It is thus a feature of at least one embodiment of the invention to permit image-guided multiple measurements of connective tissue such as a ligament for improved accuracy in ligament characterization.

These particular objects and advantages may apply to only some embodiments falling within the claims and thus do not define the scope of the invention.

### BRIEF DESCRIPTION OF THE DRAWINGS

FIG. 1 is a side elevational view of a human ankle and foot showing an example sensor/stimulator unit constructed according to one embodiment of the present invention as applied proximally to a patient's Achilles tendon to induce a shear wave in the tendon through the skin to enable the measurement of shear wave speed;

FIG. 2 is a flowchart of a program executed on an electronic computer associated with the sensor/stimulator unit for calculation of shear wave velocity;

FIG. 3 is a simplified and expanded view of a tendon and sensor/stimulator unit of FIG. 1 showing the simplified waveforms obtained by the unit and their processing according to the program of FIG. 2;

FIG. 4 is a side elevational view in phantom of an alternative embodiment of the sensor/stimulator unit of FIG. 1 providing for mechanical scanning of transducer elements instead of multiple transducer elements;

FIG. 5 is a block diagram showing additional processing of the shear wave data obtained by the present invention for other tissue characterizations;

FIG. 6 is an example screen display that may be provided by the present invention for generalized display of properties of tendon-like tissue;

FIG. 7 is an alternative embodiment of the sensor stimulator of FIG. 1 providing for simultaneous multidimensional B-mode imaging;

FIG. 8 is an alternative display to that shown in FIG. 6 providing dynamic measurement of applied stress to tissue during use;

FIG. 9 is an alternative display to that shown in FIGS. 6 and 7 showing tracking of stresses in different layers of the tissue possible with the present invention;

FIG. 10 is a figure similar to that of FIG. 1 showing an alternate embodiment of the invention which deduces shear wave travel using skin-mounted accelerometers;

FIG. 11 is a perspective view of a handheld embodiment of the present invention for use in assessing connective tissue tension in surgical procedures such as total knee arthroplasty;

FIG. 12 is a block diagram of the principal components of the embodiment of FIG. 11;

FIG. 13 is an example display provided by the handheld embodiment of FIG. 11 showing measured ligament force on a lateral and medial side of a joint;

FIG. 14 is a figure similar to that of FIG. 13 showing a display showing variations in force with different angulations of the joint;

FIG. 15a is a simplified representation of a force-limiting coupling that may be used in the handheld embodiment of FIG. 11 to prevent inadvertent flexure of the ligament during the measurement and provide feedback so data acquisition occurs only within the acceptable force range (i.e., transverse force window);

FIG. 15b is a diagram of measured threes on the sensor and stimulator of the embodiment of FIG. 11 showing automatic data acquisition within a transverse force window;

FIG. 16a is an example display similar to that of FIGS. 12 and 13 showing separate measurements of ligament force in different longitudinal positions displaced in a range of anterior and posterior positions;

FIG. 16b is a front elevational view of a distal end of the embodiment of FIG. 11 showing an array of sensors for producing the measurements of FIG. 16a;

FIG. 17 is a simplified perspective view of the embodiment of FIG. 11 used with a position sensing system showing in inset a display similar to the displays of FIGS. 13, 14 and 16a providing assistance in interactive location of the handheld unit of FIG. 11. with respect to fixed references of the joint bones;

FIG. 18a is an alternative embodiment of the invention with the stimulator and probe supported on a plastic prosthesis that may be temporarily fit within the joint; and

FIG. 18b is a figure similar to FIG. 11 showing the prosthesis of FIG. 18a in position within the joint for ligament measurement.

### DETAILED DESCRIPTION OF THE PREFERRED EMBODIMENT

### First Embodiment

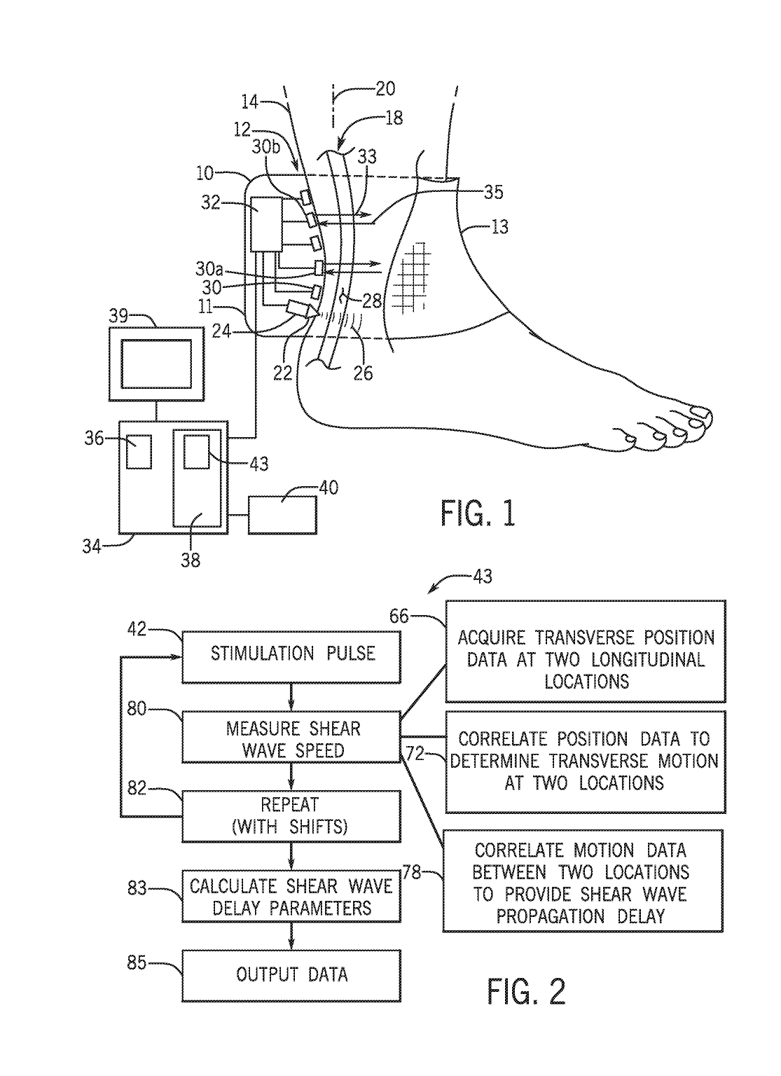

Referring now to FIG. 1, a measuring unit 10, constructed according to one embodiment of the present invention, may provide a housing 11 having a front face 12 sized and shaped to place against skin 14 of a patient proximate to a tendon or ligament (henceforth connective tissue 18) extending along a longitudinal axis 20. The housing 11 may be held against the skin by means of an elastic cuff 13 or the like. For example, the housing 11 may be applied with its front face 12 against the rear of a patient's ankle adjacent to and vertically aligned with the Achilles tendon.

Exposed at a lower edge of the front face 12 of the housing 11 is a stimulator probe 22 attached to piezoelectric element 24. The stimulator probe 22 is shaped to press against the skin overlying the Achilles tendon to conduct force from the piezoelectric element 24 through the skin to apply a periodic transverse stimulation pressure 26 to the tissue 18 inducing a longitudinally traveling shear wave pulse 28 traveling vertically upward therefrom. As is understood in the art, piezoelectric materials are those which change dimension under the influence of the electric field and thus can be used to provide mechanical motion under electrical control.

Also exposed at the front face 12 of the housing 11 are multiple ultrasonic sensors 30 arrayed generally in a vertically extending longitudinal line. In a simplest case, two spaced-apart ultrasonic sensors 30a and 30b may be positioned at predetermined locations in longitudinal separation from the stimulator probe 22 as discussed. These ultrasonic sensors 30 may emit ultrasonic waves 33 and measure returning echoes 35 in the manner of conventional ultrasound imaging transducers.

Each of the stimulator probe 22 and the ultrasonic sensors 30 communicate with a local signal processing circuit 32 which may provide signals to the stimulator probe 22 and the ultrasonic sensors 30 from electronic computer 34 and may sample and digitize the data from the ultrasonic sensors 30 for transmission to the electronic computer 34. Generally, signals from each of the ultrasonic sensors 30a and 30b provide a time series of ultrasound RF data.

The electronic computer 34 may include one or more processors 36 communicating with a memory 38 holding a program 43 as will be described below. In addition, the electronic computer 34 may communicate with the signal processing circuit 32 to send data to the stimulator probe 22 and ultrasonic sensors 30 and to collect data from the ultrasonic sensors 30 that may also be stored in memory 38 for processing. As will be described below, the electronic computer 34 may further change the particular ultrasonic sensors 30 selected as ultrasonic sensors 30a and 30b or may control mechanical elements to scan the ultrasonic sensors 30a and 30b in an alternative embodiment also discussed below.

The electronic computer 34 communicates with a graphics display 39 of a type known in the art as well as human input controls 40 such as a keyboard, mouse, touchscreen, or the like, allowing a human operator to input data and control the acquisition of data using the present device.

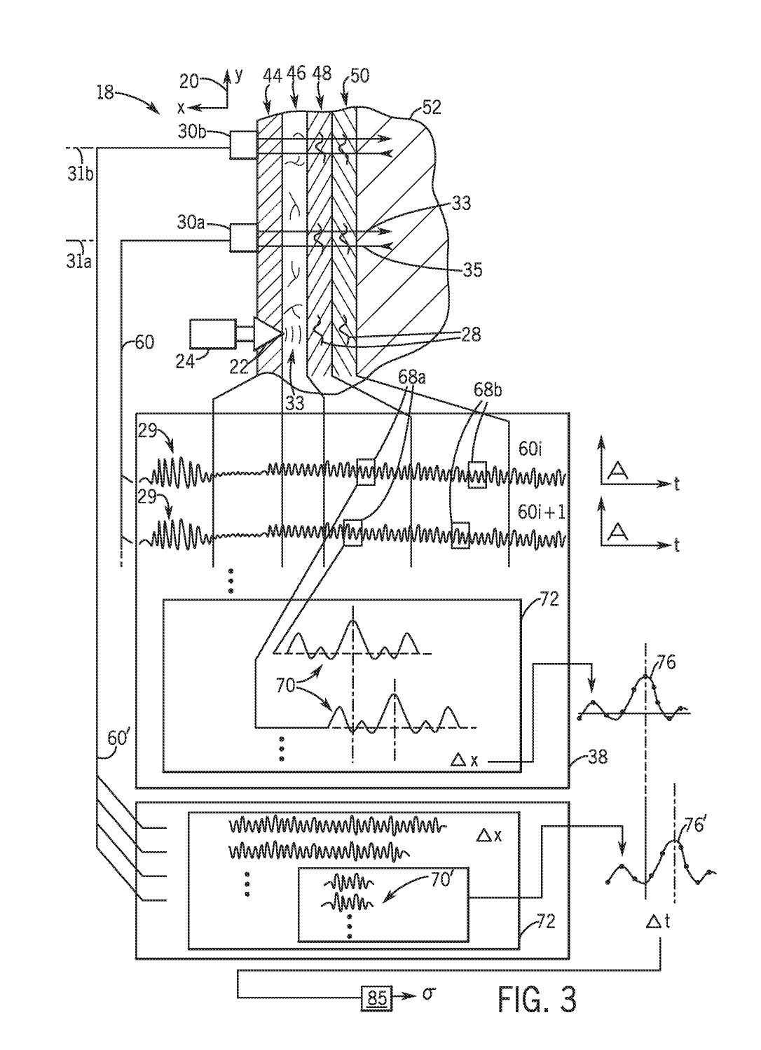

Referring now also to FIGS. 2 and 3 as well, the measuring unit 10 operating with the electronic computer 34 may make repeated measurements of shear wave propagation speed, for example, at a repetition frequency of greater than 20 hertz and at rates from 50 to 100 hertz. At the beginning of each periodic measurement, stimulation pressure 26 is applied to the tissue 18 as indicated by process block 42, for example, providing an impulse signal to the piezoelectric element 24 (for example, a short square wave pulse or sinc pulse) and then quieting the piezoelectric element 24 for the remainder of the measurement. It will be understood that this pulse provides a range of frequencies of stimulation as a result of its short duration. Typically, the duty cycle of the transducer operating in this fashion will be less than ten percent, meaning that the piezoelectric element 24 remains quiet without motion for most of the time during operation of the measuring unit 10. However, other duty cycles including a 50 percent duty cycle may be used.

The transverse stimulation pressure 26 passes through a gel or ultrasound-conducting pad layer 44 then through the skin 46 and into the superficial tendon 48 (gastrocenemius) and then through the deep tendon 50 (soleus) and then into other muscle and tissue 52. Alternatively, the stimulation pressure 26 may pass strictly through the skin.

The result of the transverse stimulation pressure 26 is to produce a shear wave pulse 28 in the superficial tendon 48 (and 28′ in the deep tendon 50) traveling upwardly and longitudinally (along the Y-axis as shown) along the tissue 18. It will be appreciated that some shear waves will also pass up the other layers including the gel layer 44, skin layer 46, and muscle 52 such as may be distinguished from the pulses 28a by intensity, speed, or depth.

The shear wave pulses 28 arrive first at ultrasonic transducer 30a longitudinally displaced from the stimulator probe 22 and positioned to direct ultrasound waves 33 transversely along a first transverse axis 31a (also indicated as the X-axis) through each of the layers 44, 46, 48, 50, and 52 and receive return echo signals 35 at various points along that path, for example, from tissue interfaces and other elements providing “speckle”. The result is a series of time domain raw ultrasound radiofrequency signals 60 (shown distinguished by index i and each providing a “frame” of data) having time domain defined by portions corresponding to each of the layers 44, 46, 48, 50 and 52 and more generally providing an echo signal amplitude as a function of time reference to a corresponding timing of the emission of the ultrasound wave 33.

The shear wave pulses 28 next arrive at the second ultrasonic transducer 30b displaced longitudinally along axis 20 to a second transverse axis 31b further beyond the ultrasonic transducer 30a of first transverse axis 31a. Typically, the first transverse axis 31a and second transverse axis 31b will have longitudinal separation of 1 to 2 centimeters. Ultrasonic sensors 30a and 30b may be single-element transducers or multi-element transducers.

Like ultrasonic transducer 30a, ultrasonic transducer 30b are positioned to direct ultrasound wave 33 transversely through each of the layers 44, 46, 48, 50, and 52 and received return echo signals 35 in return providing raw ultrasound signal 60′. The acquisition of this set of echo signals 60i and 60′i is indicated by process block 66 and may be stored in memory 38 for later processing.

The signals 60 and 60′ may be acquired at an extremely high rate based on the simple transducer structure of the present invention providing in excess of at least 8000 frames per second and typically in excess of 14,000 frames per second for each transducer 30a and 30b.

It will be appreciated that for a given elapsed time after generation of the emitted ultrasound wave 33 associated with each signal 60, the signal 60 will indicate echoes returned from different depths within this tissue 18 unique to different of layers 44, 46, 48, 50, and 52 along transverse axis 31a. Accordingly, a time window 68 may be applied to each signal 60 being a predetermined fixed time delay after the excitation signal 29 of the emitted ultrasound wave 33 to isolate signal portions relevant to particular layers, For example, a time window 68a may provide for corresponding depth signal portion 70 among the different signals 60 relevant to the layer 48 and time window 68b may provide for signal portion 70 (not shown) among the different signals 60 relevant to layer 50.

Similar windows 68a and 68b may be applied to the signals 60′ to generate depth signal portion 70 for corresponding layers positioned along axis 31b.

At process blocks 72, signal portions 70 for each window 68a and 68b for each of signal 60 and 60′ are correlated (i.e., between sequentially acquired signals 60 for corresponding windows 68 of corresponding depths, and independently between sequentially acquired signals 60′ for corresponding windows 68 of corresponding depths) to determine separately the relative transverse motion of the tissue 18 along the transverse axes 31a and 31b. This transverse motion will be determined from the timeshift necessary for maximum correlation times the approximate sound speed of ultrasound transversely through the tissue 18. It will be appreciated that precision with respect to knowing the transverse displacement is not required and that the instrument may be used for revealing relative changes as well as providing absolute quantitative measurements.

Successively calculated displacements for successive signals 60 yield transverse motion signals 76 being a set of displacements over time indicating the transverse motion of the particular tissue element of layer 48 or 50 at axis 31a. Similarly, successively calculated displacements for successive signals 60′ yield transverse motion signals 76 being a set of displacements over time indicating the transverse motion of the particular tissue element of layer 48 or 50 at axis 31b.

The motion signals 76 and 76′ describe the evolution of transverse tissue deformation caused by the propagation of shear wave pulse 28 as it propagates along the tissue 18. Accordingly, two motion signals 76 and 76′ may be then compared, as indicated by process block 78, to determine a Δt value being equal to time it took the shear wave pulse 28 to travel between the axes 31a and 31b. It will be understood that this Δt value is inversely proportional to the shear wave speed and that shear wave speed may be determined simply by knowing the longitudinal separation between axes 31a and axis 31b.

The processes of process block 66, 72 and 78 makeup process block 80 shown in FIG. 2.

The speed of propagation of a shear wave is typically much lower than the speed of propagation of a compression wave through the tissue 18, for example, with compression waves traveling at 1800 to 2000 meters per second and shear waves traveling from approximately 10 to 100 meters per second depending on the stress applied to the tendon. Nevertheless, it will be appreciated that high spatial and temporal accuracy is necessary to resolve shear wave speed differences over the short distance of the separation of the ultrasonic sensors 30 at axes 31a and 31b. Shear wave speed is substantially more sensitive to tissue stress (above speeds of over approximately 15 meters per second) than measurement of compression wave speed.

This measurement of shear wave propagation speed may be repeated by looping back to process block 42 to apply a new stimulation pulse between measurements of process blocks 66, 72 and 78 indicated generally by process block 82. The shift at process block 82 is optional and the repeated measurements may be made at the same location, for example, under dynamic loading.

During or subsequent to the process of process blocks 42, 80, and 82, stress measurements may be determined from the shear wave speeds as indicated by process block 83. In one embodiment, stress on the tissue 18 may be derived according to the following equation modeling the tissue 18 as a Timoshenko beam as follows:

$$v = {( \frac{{k}^{′} μ + σ}{ρ} )}^{\frac{1}{2}} \qquad ( 1 )$$

where ν is shear wave longitudinal speed determined by knowledge of the separation of the axes 31 discussed above;

k′ is a shear correction factor empirically determined for a particular tissue and geometry being studied;

μ is the shear elastic modulus (that may be determined empirically for a particular tissue type);

ρ is the effective tissue density (generally known for a particular tissue type and its surroundings); and

σ is the axial stress on the tissue.

The inventors have determined that axial stress dominates this equation (1) when even moderate stresses are applied to tendon tissue allowing the equation to be simplified to:

σ=ρν² (2)

Alternatively, a measurement may be made using equation (1) at zero axial stress to deduce a constant

$$( \frac{{k}^{′} μ}{ρ} )$$

to be used in extracting axial stress.

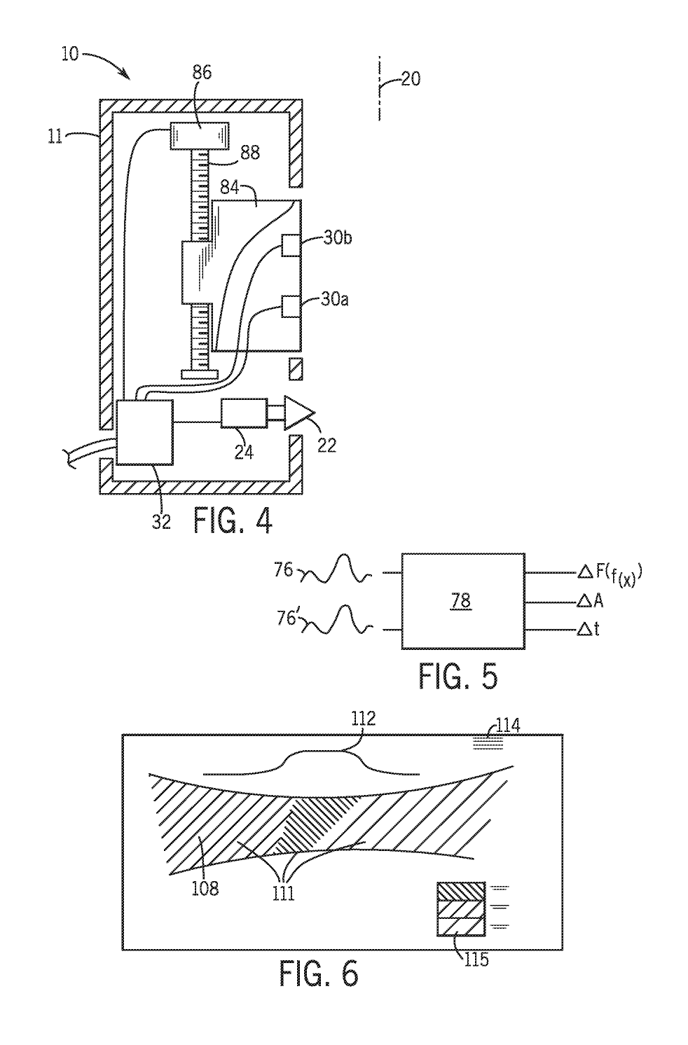

This value may then be output as indicated, for example, by process block 85 of FIG. 4. In addition to or alternatively various shear wave speed derived parameters may be output including but not limited to tension, shear wave delay, shear elastic modulus and density. These latter two measures may be determined simply by making assumptions about shear wave speed (for example, by empirical measurement) and solving equation (1) for different variables. The former measure of tension can be deduced from stress with the knowledge of cross-sectional area of the ligament as will be discussed below

It will be appreciated that the present technique may provide not only quantitative axial stress but qualitative axial stress in cases where only qualitative indications of stresses are required, for example, as provided by the shear wave speed itself Such measures may be useful for qualitative displays of tissue properties.

Referring now to FIGS. 1 and 2, at the conclusion of each measurement of process block 80, and prior to the repetition of process block 82, the location of the ultrasonic sensors 30a and 30b may be shifted along the tissue 18 (with or without shifting the stimulator probe 22) to measure stress-related properties at different longitudinal portions of the tissue 18. These properties may include changes in stress in the tissue or changes in the other properties described above. In cases where the stress along the tissue 18 may be assumed to be substantially constant, insight and other tissue properties such as elastic modulus may be better revealed.

Referring to FIG. 1, this movement of the location of ultrasonic sensors 30a and 30b may be performed by simply selecting among different pairs of ultrasonic sensors 30 in a longitudinal array of ultrasonic sensors 30 only some of which are activated. Alternatively, as shown in FIG. 4, a pair of individual ultrasonic sensors 30a and 30b may be mounted on a movable carriage 84 translated by electrically controlled actuator 86 such as a stepper motor or the like driving a lead screw 88 to physically translate the ultrasonic sensors 30 along the axis 20. This actuator 86 may be controlled by the computer 34 through the signal processing circuit 32.

Referring now to FIG. 5, the motion signals 76 and 76′ as described above may be further analyzed to determine a change in the shape of the shear wave pulse 28 as it progresses through the tissue such as may reveal a damping effect of the tissue, for example, caused by tissue viscosity or the like. Accordingly process block 78 may be supplemented to provide not only a Δt value indicating the propagation delay of the shear wave pulse 28 through the tissue 18 but also a decrease in amplitude or a change in spectral content represented by the shear wave pulse 28, for example, produced by a Fourier transform of the motion signals 76 and 76′ or a similar measure such as power spectra difference.

Referring to FIG. 8, repeated execution of process blocks 42, 80 and 83 (without necessarily shifting the position of the ultrasonic sensors 30) may be used to create a dynamic stress plot 100 showing a representation of the stress on the tissue 18 over time, for example, with a patient walking or performing other activity. This stress plot 100 may be displayed on the display 39 for analysis, for example, together with a video image 102 of the patient showing a frame of the patient activity such as walking coordinated with a cursor 104 moving over the stress plot 100 to a corresponding time.

Referring now to FIGS. 6, 7 and 9, in one embodiment, the measuring unit 10 may make use of an array 105 of ultrasonic sensors 30 having columns extending along the longitudinal axis 20. Depth information is obtained using the known speed of ultrasound transmission from to determine the depth at which tissue structures generated the ultrasound echo. This depth information is obtained for each element, allowing for the acquisition of a two-dimensional B-mode image 108. This B-mode acquisition may occur before or after the processing of shear wave propagation speed measurement. Portions 111 of the B mode image may be shaded or colored to reflect tissue properties determined by the present invention, for example, by matching the data determined from process block 85 with a shifting of the ultrasonic sensors 30a and 30b corresponding to longitudinal location in the B-mode image. This shear wave speed-related data may also be displayed in addition to or alternatively along only a single dimension per plot line 113, and quantitative information 114 may also be provided as derived above with respect to process block 82 of FIGS. 2 and 5. A key 115 may be provided to decode the shading to quantitative values or ranges.

During the determination of shear wave speed, ultrasound is collected at a small number of (two or more individual ultrasonic transducers) 30 may be actuated to obtain high frame rate information. Alternatively, planar wave imaging may be used to achieve for high-frame rate data acquisition.

Referring now to FIG. 9, the ability to rapidly measure axial stress in multiple tissue layers (for example, tissue layers 48 and 50) of FIG. 3 allows simultaneous display of axial stress or a similar quantity for each of the different tissue layers. In this way depth-related differences in tissue properties may be determined, for example, showing a deviation between axial stress 110 for superficial tendon layers versus axial stress 112 for deeper tendon layers.

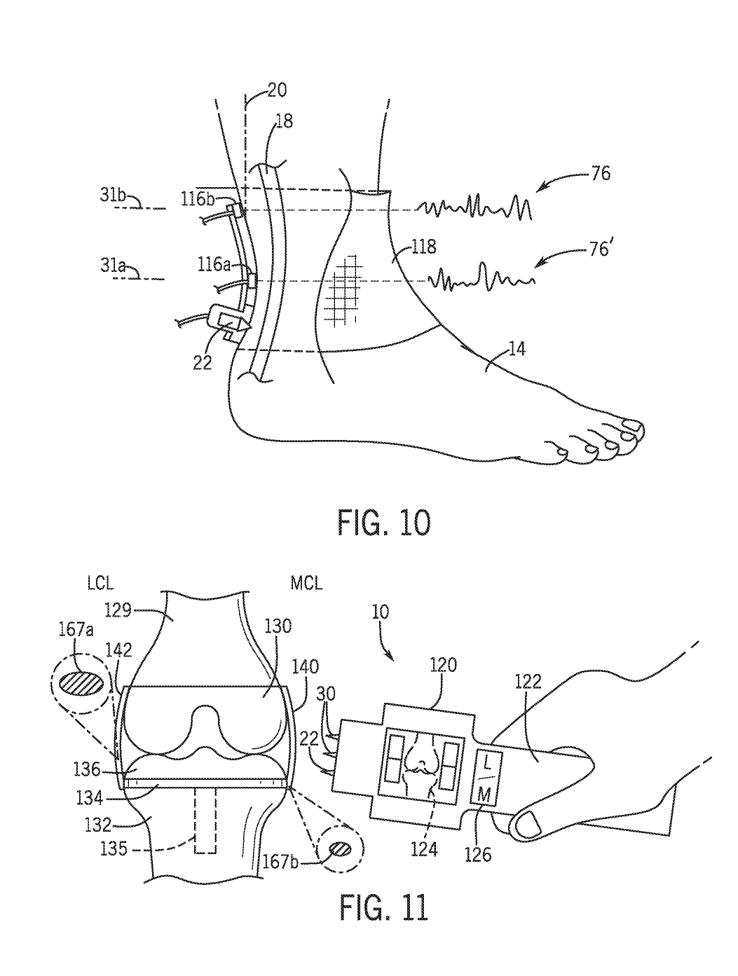

Referring now to FIG. 10, in an alternative embodiment, the ultrasound ultrasonic sensors 30a and 30b may be replaced with skin-mounted accelerometers 116a and 116b, each providing an axis of sensitivity along respective axes 31 as described above. The accelerometers 116, for example, may be microelectromechanical devices having low mass and high sensitivity, for example, the ADX L212 accelerometer from Analog Devices of Massachusetts having sensitivity of +/−2 g with the Z-axis aligned with axis 31.

A signal from the z-axis accelerometers 116a and 116b may provide motion signals 76 and 76′ as discussed above and these motion signals 76 and 76′ may be processed as described above, for example, with respect to FIGS. 2, 3, and 5, to provide the measurements also discussed above.

In this respect, it will be understood that ultrasonic sensors 30 and accelerometers 116 both provide a function of motion sensors of transverse motion. Generally, the signals from the accelerometers 116 will indicate a second derivative of position of the tissue 18 and will measure a position that is a combined effect of shear waves moving through multiple layers of tissue 18 as opposed to the ultrasonic sensors 30 which may distinguish between motion at different layers and measure motion directly. This second derivative signal can be integrated twice to provide a position or may be used directly as if the acceleration signals were position signals, either of which will provide an indication of shear wave speed.

In one embodiment, the accelerometers 116 may be precisely spaced in a supporting housing (not shown) including damping elements to prevent the communication of vibration through the housing between the accelerometers 116 or between the stimulator probe 22 and the accelerometers 116.

Close coupling of the accelerometers 116 to the skin of the patient may be provided a pressure sensitive adhesive (not shown) or by an elastic cuff 118 holding the accelerometers 116a and 116b in a fixed longitudinal separation and biased against the skin. The same cuff 118 may support the stimulator probe 22 operating as discussed above to be spaced from the lower accelerometer 116b. The material of the cuff 118 may be selected to provide very little coupling between the stimulator probe 22 and the accelerometers 116, for example, by using a material that is relatively compliant and that has high damping measured in the longitudinal direction.

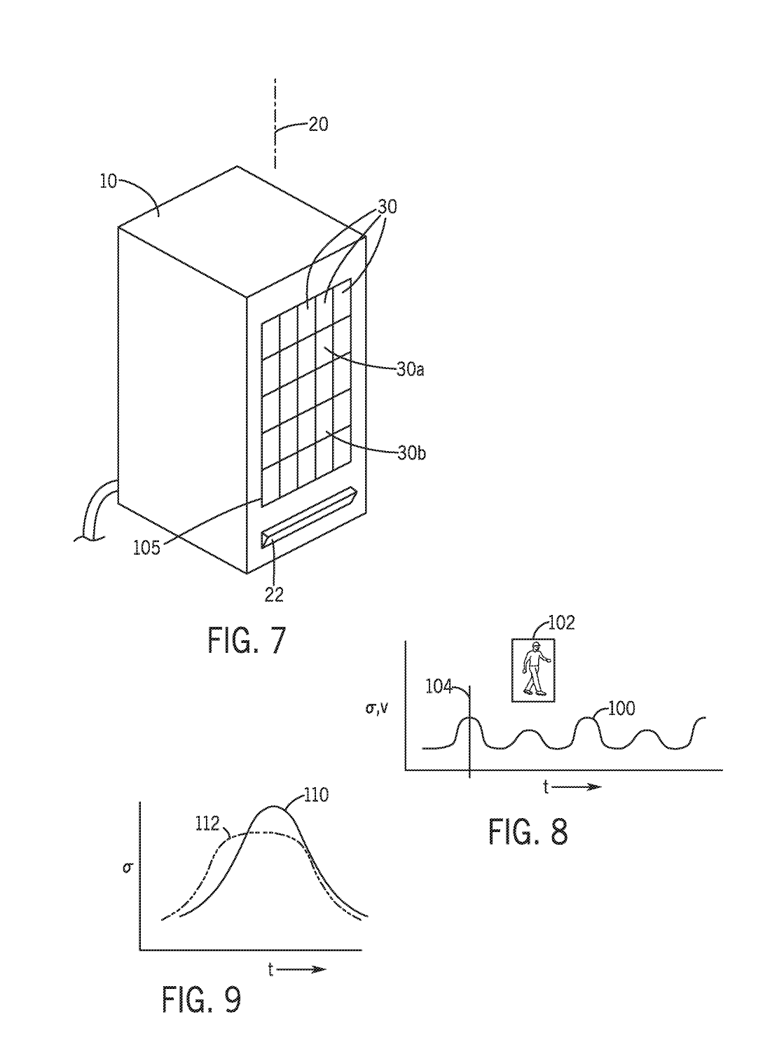

Multiple accelerometers 116 may be arrayed longitudinally along axis 20 to provide for the ability to make shear wave measurements at different longitudinal locations along the tissue 18 as discussed above with respect to the ultrasound ultrasonic sensors 30. Alternatively, two accelerometers 116a and 116b may be mounted as shown in FIG. 4 in place of the ultrasound ultrasonic sensors 30a and 30b and moved mechanically along the surface of the skin, coupled to the mechanical carriage 84 by a motion damping material and by using a lubricating medium between the face of the accelerometers 116 and the skin to permit this motion. Multiple accelerometers 116 may also be arrayed transversely, for example, as shown in FIG. 7 to allow measurements along different longitudinal axes that are transversely separated.

In other respects, this embodiment may make use of the components and techniques described above with respect to the non-contact sensors of an ultrasonic transducer system or laser system.

### Second Embodiment

Referring now to FIG. 11, the present invention may be contained in part in a handheld housing 120 having a handle portion 122 that may be supported and positioned by a user's hand. The housing 120 may provide a display 124, for example, an LCD display, positioned to be visible during use to convey settings and measurement data to the user. Positioned and exposed at a distal end of the housing 120 are the sensors 30 and stimulator probe 22 extending therefrom and generally separated along a vertical, longitudinal axis as is discussed above with respect to FIG. 1. The sensors 30 may in this example be accelerometers. One or more human input controls 126, for example, tactile buttons or the like may be exposed on the outer surface of the housing 120 to allow changing of the mode of operation of the unit as will be described.

In this handheld configuration, the measuring unit 10 may be used intraoperatively, for example, during a total knee arthroplasty procedure or other similar procedures. In total knee arthroplasty, a distal end of the femur 129 is resected and fit with a femur prosthesis componenet 130. A similar resection of the tibia 132 may be performed and a corresponding prosthesis baseplate 134 installed over the resected region, for example, fixed by a keel 135 extending axially into the tibia. A plastic liner 136 is then fit on top of the prosthesis baseplate 134 between the prosthesis baseplate 134 and the femur prosthesis componenet 130 to provide a self lubricating interface between the liner 136 and the femur prosthesis componenet 130.

During installation of the prosthesis, the measuring unit 10 may be manipulated to position the distal end against and aligned with superficial medial collateral ligament 140 or alternatively against the outer surface of the lateral collateral ligament 142 to measure the force affecting these ligaments for balancing the knee joint.

Generally will be understood that measurements from other sides including the inside of these ligaments 140 and 142 may also be taken.

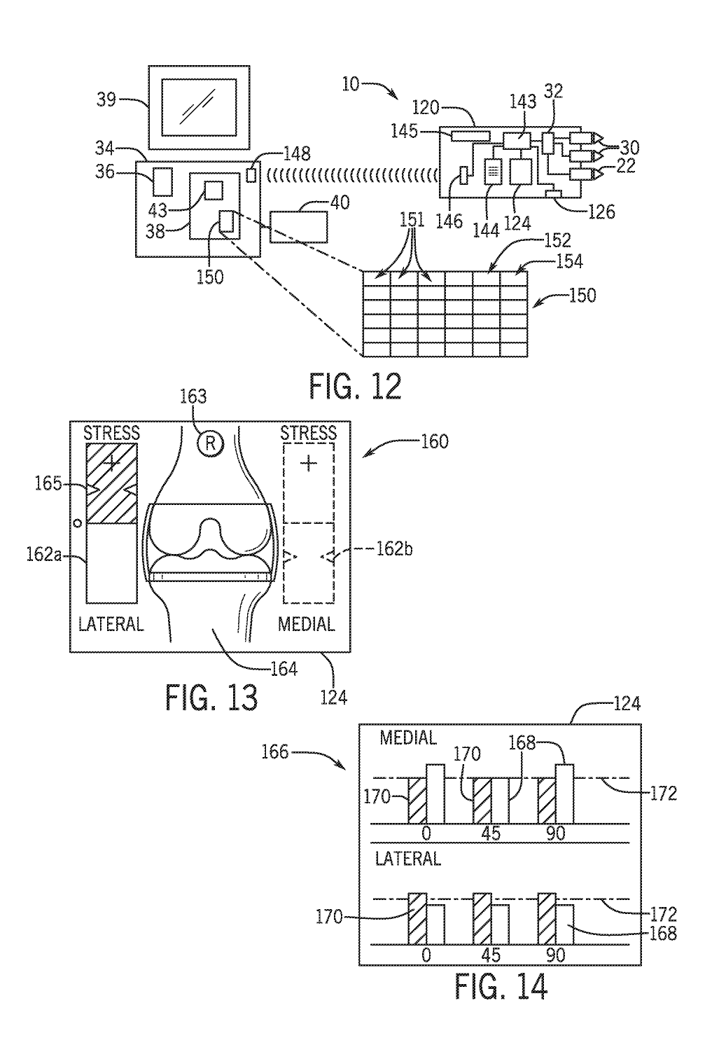

Referring now to FIG. 12, the housing 120 may also hold the signal processing circuit 32 communicating with the sensors 30 and stimulator probe 22 and with an internal processor 143 handling other functions of the measuring unit 10. The internal processor 143 may communicate with the display 124 and with a memory 144 holding data values as will be discussed and an internal operating program implementing different display modes to be discussed. Each of these components of the measuring unit 10 may be powered by a contained battery 145 for portable wireless use.

The internal processor 143 may also receive data from the human input controls 126 and may communicate via a wireless link circuit 146 (for example, Bluetooth or Wi-Fi) with a corresponding wireless link circuit 148 associated with the computer 34 as discussed above.

Generally, it will be appreciated that various functions to be described below may be freely allocated between the computer 34 and the internal processor 143 of the measuring unit 10, and similar allocation can be performed with respect to the location of the human machine interface and storage of data. Accordingly, the following description which associates particular functions with a particular location should not be considered a limitation to operation unless context so requires.

Referring now to FIGS. 11, 12, and 13, the measuring unit 10 may desirably be used to evaluate the stress or tension in the ligaments 140 and 142 against a desired or a normal stress or tension. In this regard, the memory 38 of computer 34 may hold a data table 150 providing empirically determined desired stress or tension values of these two ligaments 140 and 142 indexed according to fields 151 providing characteristics of the individual patient including, for example: age, gender, weight, height, activity level, and physical conditioning. This data table 150 may also hold ligament characteristics, for example, aspect ratio and cross-section or the like further indexed according to particular type of ligament (e.g., medial, lateral).

The physician may enter one or more of these index values into the computer 34 to obtain a corresponding desired or normal stress or tension value 152 for a particular ligament 140 or 142 and, for example, a cross-sectional area 154 of the ligament used for the conversion between stress and tension. Other desired or normal values including shear wave delay, sheer elastic modulus, density and the like may alternatively or in addition be provided. These desired or normal values will henceforth be termed baseline values.

The baseline values may be communicated by the wireless link circuits 148 and 146 to the processor 143 to be stored in the memory 144 and may be used to generate shear wave propagation measurements as described above to produce output data displayed in first display mode 160 shown in FIG. 13 providing an indication of a comparison of actual measured values of the ligament 140 or 142 and the baseline values for those ligaments. In one example, this first display mode 160 may provide for left and right graphic error bars 162a and 162b positioned on either side of a simplified image 164 of the joint associated with the ligaments 140 and 142. The joint depiction may be labeled, for example, with a label 163 indicating whether it is the right or left side of the patient and the error bars 162a and 162b may be positioned appropriately on the lateral and medial sides of the simplified image 164, such positions which may also be labeled by text. In this example, each error bar 162 may provide a vertically extending rectangle divided into an upper and lower portion associated with plus and minus error values being the difference between actual measured values and baseline values. These upper and lower portions may be labeled with plus and minus symbols and shaded appropriately.

The actual measured values (typically a user selected stress or tension) may be indicated by a vertically sliding measurement arrow 165 displaced from a center of the error bar (labeled zero) according to the difference between the baseline value and the actual measured value of the ligament 140 or 142. In this way, the physician may quickly determine any needed adjustment of the ligament through release or adjustment according to known surgical techniques.

In the display of FIG. 13, a single error bar 162 may be given focus and the other error bar 162a, for example, grayed out to indicate the particular ligament 140 or 142 which is being measured. Previously measured values and their baselines may nevertheless be visible in both the focused and unfocused error bars 162 so that ready comparison can be made with respect to current ligament tensions. A switching between medial and lateral measurements may be provided by activation of a human input control 126 such as a button.

An additional numerical or graphical display (not shown) can provide a difference in either load measures (e.g., tension or stress) between the medial and lateral ligaments 140 for improved assistance in balancing. As noted, the displayed values may be expressed in a variety of different forms including, for example, stress or tension, the latter of which will vary for a given stress according to the cross-sectional areas 67 of the ligaments 140, 142. In this regard, referring briefly again to FIG. 11, ligament 140 will generally have a smaller cross-sectional area 167b, and hence a much higher aspect ratio, than the cross-section area 167a of ligament 142. The values of these cross-sectional areas 67 may be provided by the data table 150 based on empirical or modeled data and be transmitted to the memory 144 along with the baseline values used for conversions between tension and stress. A particular display expression may be preselected according to clinical evidence or selected by the physician.

Referring now to FIG. 14, in a second display mode 166, a record may be kept of measured ligament load values for each ligament 140 and 142 at different limb angles as the leg or other joint is moved between full extension (zero degrees) and partial flexion (90 degrees) so that the stored values can be viewed side-by-side for comparison. As depicted, current measured forces 168 may be provided at each of these flexion angles together with a previously measured set of forces 170, for example, depicted as different colored bars of a bar chart having a pair of bars for each flexion angle. A baseline value 172 or average value (or both) may also be projected across these bars for reference. As noted, this display mode 166 assists in assessing changes in ligament force as the knee joint is flexed.

Referring now to FIGS. 16a and 16b, in a third display mode 174, a joint image 176 may be provided along the sagittal plane with three measured force bars 175 positioned respectively aligned with the joint, displaced laterally in an anterior and posterior direction to provide different measures of the force of a given ligament 140 or 142 at its midline, anterior edge, and posterior edge respectively. These three measures show how forces are distributed within a single ligament 140 or 142 to assist in balancing forces at different leg flexion angles. The underlying measurements may be made with separate repositioning of the handheld measuring unit 10 or by use of an array of sensors depicted in FIG. 16b in which two laterally extending rows and three longitudinally extending columns of sensors 30 are separated both laterally and longitudinally along individual longitudinal axes 178. A single or multiple stimulator probes 22 may cover this lateral range.

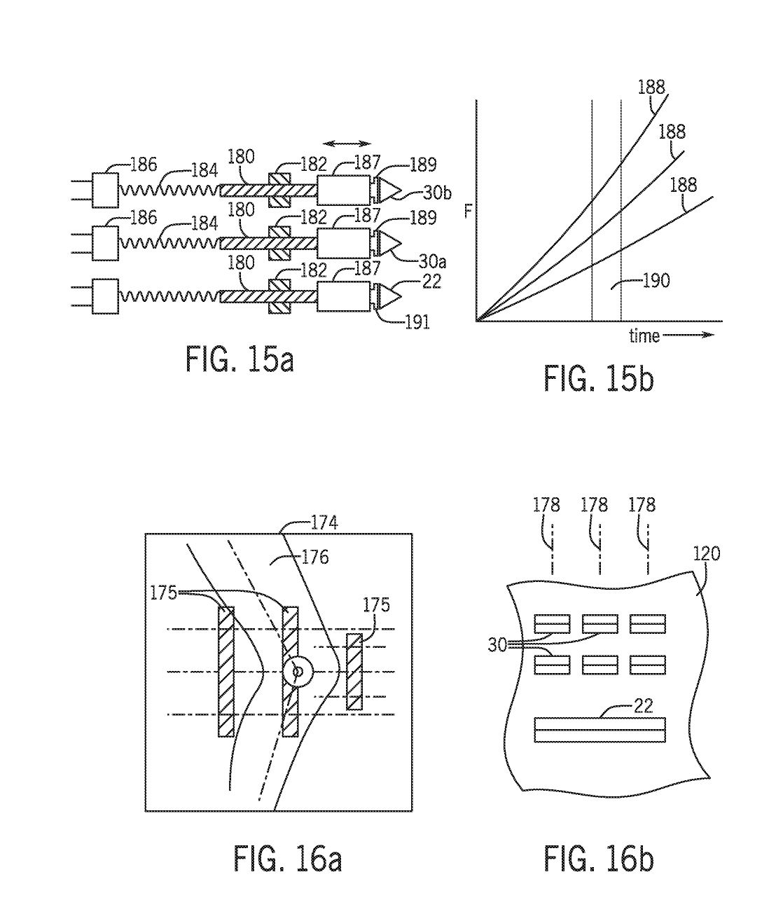

Referring now to FIGS. 15a and 15b, the handheld configuration may guard against or compensate for inadvertent tensioning of the ligaments 140 and 142 caused by pressure between the handheld measuring unit 10 and that ligament by means of one or more force-limiting couplers limiting or controlling the steady-state force between the handheld measuring unit 10 and the ligament 140 or 142 to a predetermined amount or limit. The steady-state transverse force on the ligament 140 or 142 should be distinguished in the case of the stimulator probe 22 from the brief excitation used to generate shear waves whose steady-state value may be zero.

In one embodiment of the force-limiting coupler, each of the sensors 30a and 30b and stimulator probe 22 may be mounted on independently movable transverse axial slides 180 sliding with respect to guides 182 fixed with respect to the housing 120. Each slide 180 may be spring-biased by a compliant (low k-value) spring 184 (for example a helical spring or an air spring) providing a relatively constant but low force over the range of motion of the slides 180 both to limit the applied force and prevent minor variations in relative positioning of the handheld measuring unit 10 and the ligament 140 from imparting substantially different transverse deflections (and hence increased axial strain) to the ligaments 140 and 142. In one embodiment, the springs 184 may extend between an end of each slide 180 and a corresponding force transducer 186 (for example, load cells), the latter fixed with respect to the housing 120. In this configuration, the actual transverse force between a given sensor 30 or stimulator probe 22 and the ligament 140 or 142 (which rises slightly with compressions of the spring 184) may be measured. The measurement may be used to provide guidance to the physician in the application of pressure or may be tracked to automatically obtain ligament measurements when the transverse forces are within a predetermined value. Thus, as shown in FIG. 15, separate forces 188 on the ligament from each of the sensors 30a, 30b, and 30c may be tracked over time and measurement of shear wave speed may be triggered when these forces pass through a predetermined force window 190 to provide for consistent measurement of transverse forces. When force transducers 186 are used they may also be used without the springs 184 and simply provide guidance to the physician/user with respect to the force being applied so that consistent measurements may be made. The display of force may be simply a status light that shows “green” when the force is in an acceptable range and “red when the force is outside the acceptable range. In one embodiment, shear wave speeds would not be displayed when these forces are outside the acceptable range as to prevent erroneous measurements.

Desirably, the high-speed vibrations created by and sensed by the stimulator probe 22 and sensors 30 are isolated from the compliant springs 184 using small weights 187 positioned between the slides 180 and, for example, the accelerometers 189 of the sensors 30 or between the slides 180 and electromagnetic transducer 191 of stimulator probe 22. It will be appreciated that a force-limited coupling can also be obtained using an active servo system that moves the sensors 30 and stimulator probe 22 in response to sensed forces, for example, sensed by force transducers 186.

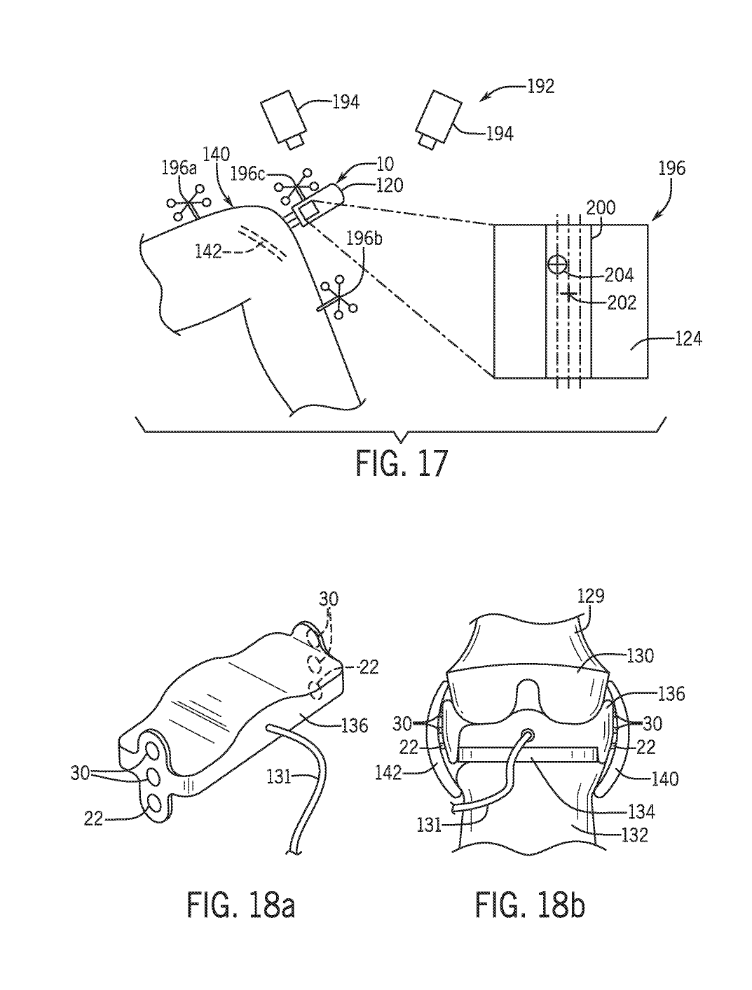

Referring now to FIG. 17, it is contemplated that the handheld measuring unit 10 may work in conjunction with stereotactic positioning systems 192, for example, in one common implementation, employing cameras 194 and optical fiducial elements 196a 196b and 196c, for example, attached respectively to the femur, tibia, and the housing 120 of handheld measuring unit 10. The cameras 194 allow precise location and determination of the orientation of the fiducial elements 196 through triangulation.

In this case, the absolute position of the handheld measuring unit 10 with respect to the bones of the femur and tibia may be determined and hence with respect to the location of the ligaments 140 and 142 so that the display 124 may be operated in a fourth display mode 198 to provide an actual or simplified image 200 of the ligament 140 or 142 together with a desired location crosshair 202 of the measurement. An actual position and orientation of the handheld measuring unit 10 is also provided by a secondary crosshair 204. The operator may then align the handheld measuring unit 10 by aligning the crosshair 202 and crosshair 204, this process improving location of the measurement of the ligaments 140 and 142, for example, as guided by medical imaging. The desired location crosshair 202 may be varied, for example, along an anterior-posterior or superior-inferior range in the sagittal plane to obtain a sequence of readings needed for the display of FIG. 16 discussed above.

In other respects and as would be generally understood by those of skill in the art from this description, this embodiment may make use of the components and techniques described above with respect to the first embodiment and below with respect to the third embodiment to be described.

### Third Embodiment

Referring now to FIGS. 18a and 18b, the sensors 30 and stimulator probe 22 may be incorporated into a temporary plastic liner 136 (for example, serving the purpose of the liner 136 discussed with respect to FIG. 11). At least two sensors 30 and a stimulator probe 22 may face outwardly from the installed plastic liner 136 along a coronal plane beneath the respective ligaments 142 and 140 in each of the medial and lateral directions. In this configuration, load measurements on the ligaments 140 and 142 may be made simultaneously on both ligaments 140 and 142 during a trial fit of a liner 136. An electrical conductor 131 may allow signals to and from the sensors 30 and stimulator probes 22 to be communicated to a remote unit providing other elements of the handheld measuring unit 10 discussed above. Alternatively, the electrical conductor 131 may be replaced with a wireless communication circuit, for example as discussed in FIG. 12. The sensors 30 may be any of piezoelectric sensors or accelerometers as discussed above or may be non-contact type laser sensors employing Doppler shift or triangulation to make high-speed measurements of transverse motion without contact or deflection of the ligaments 140 and 142 by the sensors 30.

In other respects, and as would be generally understood by those of skill in the art from this description, this embodiment may make use of the components and techniques described above with respect to the first embodiment and above with respect to the second embodiment.

In each of these embodiments, a sterile sleeve may be applied over the sensors 30 and stimulator probe 22 to prevent the transfer of pathogens or damage to the device. Alternatively components can be manufactured of chemically sterilizable materials.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “a controller” and “a processor” can be understood to include one or more microprocessors that can communicate in a stand-alone and/or a distributed environment(s), and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

“Diameter” as used herein should not be understood to require a cylindrical or circular element but to simply describe a diameter of a circumscribing cylinder closely conforming to the element.

As used herein, ligament may refer generally to any connective tissue including tendons, ligaments, and muscles, The terms “load value”, “load measurement”, and “load measure” are intended to generally describe measurements of force and load including but not limited to stress or tension.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications are hereby incorporated herein by reference in their entireties.

1. A device for measurement of forces in connective tissue extending along a longitudinal axis, the device comprising:
a housing supportable by a hand of an individual for manual positioning of the housing;
a stimulator probe supported by the housing including an actuator adapted to apply a transverse stimulation to the connective tissue to generate a shear wave that travels longitudinally along the connective tissue;
a first motion sensor supported by the housing and detecting the shear wave in the tissue caused by the stimulator probe and along a first transverse axis at a first longitudinal position;
a second motion sensor supported by the housing and detecting the shear wave in the tissue caused by the stimulator probe and along a second transverse axis at a second longitudinal position further from the stimulator probe than the first motion sensor so that the shear wave from the stimulator probe passes the first motion sensor before arriving at the second motion sensor; and
a processing circuit operating to:
(a) receive a signal from the first motion sensor to provide a first transverse motion signal indicating first transverse movement of the tissue at the first transverse axis over time;
(b) receive a signal from the second motion sensor to provide a second transverse motion signal indicating second transverse movement of the tissue at the second transverse axis over time;
(c) compare the first transverse motion signal to the second transverse motion signal to determine a travel time of passage of the shear wave between the first and second longitudinal axes; and
(d) process the travel time to output a value functionally related to the connective tissue stress; and
wherein the stimulator probe, the first motion sensor and the second motion sensor are exposed at one end of the housing to permit stimulation of the connective tissue and to provide the output when positioned in communication with the connective tissue during surgical procedures.

2. The device of claim 1 wherein the output value provides a load measure of at least one of longitudinal stress or tension in the connective tissue.

3. The device of claim 1 wherein the processing circuit includes an electronic memory holding a data value indicating a desired preload value for the connective tissue and further including a display indicating a deviation between the desired preload value and the load measure.

4. The device of claim 2 further including an electronic memory communicating with the processing circuit and holding a database indexable by index values including at least one of age, gender, ethnicity, weight, height, and activity level providing a desired preload value for the connective tissue and further including a human machine interface for receiving the index values to identify the desired preload value for the connective tissue.

5. The device of claim 4 wherein the database provides geometric information about the connective tissue for a conversion of a load measure of stress into a load measure of tension by the processing circuit.

6. The device of claim 4 wherein the database provides a first and second desired preload value for related first and second connective tissue and further includes a display wherein the display simultaneously indicates a deviation between the first desired preload value and a first load measure of a first one of the connective tissue and the deviation between the second desired preload value and a second load measure of a second one of the connective tissue.

7. The device of claim 1 wherein the first connective tissue is a superficial medial collateral ligament and the second connective tissue is a lateral collateral ligament.

8. The device of claim 1 wherein the processing circuit includes an electronic memory holding a data value indicating a measured load value for a limb associated with the connective tissue in at least two different positions and further includes a display simultaneously indicating the measured load values for the at least two different positions.

9. The device of claim 8 wherein the processing circuit may further display on the display simultaneous information indicating a deviation between desired preload values and the load measures for the at least two different positions.

10. (canceled)

11. The device of claim 1 wherein the stimulator probe is attached to the housing with a force-limiting coupling maintaining a steady-state force between the stimulator probe and the connective tissue.

12. The device of claim 11 wherein the force-limiting coupling is a spring-biased slide mounting.

13. The device of claim 11 wherein the first motion sensor and second motion sensor are contact-type motion sensors attached to the housing with a force-limiting coupling.

14. The device of claim 1 wherein the first motion sensor and second motion sensors are non-contact type motion sensors.

15. The device of claim 1 including a position sensor and a display indicating a location of the housing with respect to the connective tissue.

16. The device of claim 15 wherein the processing circuit indicates a sequence of targets on the display to which the location of the handheld housing may be matched to provide a set of measurements of the connective tissue in different locations.

17. The device of claim 1 wherein the stimulator probe, first motion sensor, and second motion sensor, are contained in a housing supported by a spacer fitting between a femoral component and a tibial component of a knee prosthesis.

18. The device of claim 17 wherein the spacer supports a stimulator probe, first motion sensor, and second motion sensor on both of opposed medial and lateral sides of the spacer for simultaneous measurement of medial and lateral collateral ligaments.

19. The device of claim 1 further including an array of motion sensors providing at least two laterally-spaced motion sensors and at least three longitudinally-spaced motion sensors and wherein the processing circuit selects the first and second motion sensors from among the longitudinally-spaced motion sensors to vary at least one of longitudinal and lateral positions of the first and second transverse axes with respect to the tissue between measurements to provide the value functionally related to delay travel time through at least one of different longitudinal and lateral segments of the tissue.

20. A method of joint replacement comprising the steps of:
(a) resecting at least a portion of one bone of a joint for installation of a prosthesis providing a joint interface; and
(b) making measurement of a value related to connective tissue stress associated with the connective tissue of the joint by:
(i) applying a transverse stimulation to the connective tissue at a first point to generate a shear wave that travels longitudinally along the connective tissue;
(ii) detecting a first transverse motion of the tissue along a first transverse axis at a second point along a longitudinal axis including the first point;
(iii) detecting a second transverse motion of the tissue along a second transverse axis at a third point along the longitudinal axis including the first and second points;
(iv) comparing the first transverse motion to the second transverse motion to determine a travel time of passage of the shear wave between the first and second longitudinal axes; and
(v) processing the travel time to output a value functionally related to the connective tissue strain.
